(19) United States
(12) Patent Application Publication
(10) Pub. No.: US 2025/0042943 A1
(43) Pub. Date: Feb. 6, 2025

(54) FAP DETECTION

(71) Applicant: THE UNIVERSITY COURT OF THE UNIVERSITY OF EDINBURGH, Edinburgh (GB)

(72) Inventors: Ahsan AKRAM, Edinburgh (GB); Alicia MEGIA-FERNANDEZ, Ciudad Real (ES); Layla MATHIESON, Linlithgow, West Lothian (GB)

(21) Appl. No.: 18/717,317

(22) PCT Filed: Dec. 7, 2022
(86) PCT No.: PCT/GB2022/053125

§ 371 Date: Jun. 6, 2024

(30) Foreign Application Priority Data
Dec. 8, 2021 (GB) 2117727.4

(51) Int. Cl. C07K 7/06; G01N 21/64; G01N 33/68

(52) U.S. Cl. CPC C07K 7/06; G01N 21/6428; G01N 33/68; G01N 2021/6432; G01N 2333/4706

(57) ABSTRACT

The present disclosure relates to the development of novel constructs that are specifically cleaved by fibroblast activation protein-α (FAP) and uses thereof in assays for detecting FAP. Particular constructs include a fluorescence resonance energy transfer (FRET) probe comprising a fluorescent moiety and a quencher moiety attached to a FAP cleavable peptide sequence and optionally including a solubilising moiety, which may be a polyethylene glycol (PEG) derivative.

### FIELD

The present disclosure relates to the development of novel constructs that are specifically cleaved by fibroblast activation protein-α (FAP) and uses thereof in assays for detecting FAP.

### BACKGROUND

FAP is a type II transmembrane glycoprotein which is a member of the serine protease family [1]. FAP is minimally expressed by fibroblasts in health, but is highly expressed by activated fibroblasts which can be found in the stroma of epithelial tumours [2]. Furthermore, there is increasing evidence of the role of FAP in additional fibroproliferative conditions such as Idiopathic pulmonary fibrosis, hepatic fibrosis, rheumatoid arthritis and myocardial infarction [3]-[6]. Within tumours, FAP promotes tumour growth by promoting angiogenesis and ECM remodelling [7] and facilitates the progression of tumours by suppressing the anti-cancer immune response [8]. High FAP expression is associated with poor survival, high recurrence rates and more advanced stage in several cancers, including oral squamous cell carcinoma, ovarian cancer, pancreatic ductal adenocarcinoma and non-small cell lung cancer (NSCLC) [9]-[13]. Specifically in NSCLC, FAP expression has been associated with a higher peripheral neutrophil and lymphocyte count ratio and worse overall survival [14].

FAP has both endopeptidase activity (cleaving post proline peptide bonds of non-terminal amino acids) and exopeptidase activity (cleaving peptide bonds of terminal amino acids) [15]. Closely related peptidases include the dipeptidyl peptidases (DPPs), which have exopeptidase activity, and prolyl oligopeptidase (PREP or POP), which has endopeptidase activity [16]. DPP-IV is highly expressed in many tissues [17], and PREP is a closely related peptidase which in the past has been used interchangeably with FAP [18]. As PREP has also been found to be present in the membrane of fibroblasts and is distinctive over FAP, for any assay designed to detect FAP levels, selectivity is crucial [19].

Prior studies investigating FAP as an optical probe target have focused on near infrared (NIR) fluorophores where signal can be detected with minimal intrinsic tissue autofluorescence. Li et al designed an activatable NIR fluorescent probe (ANP_{FAP}) for FAP which was composed of the NIR dye Cy5.5 and the quencher dye QSY21 which were linked by a peptide sequence which is cleaved specifically by FAP (KGPGPNQC) [20]. In murine tumour models, the probe had a higher signal in FAP expressing tumours, but the study did not demonstrate selectivity over PREP. Bainbridge et al describe a FAP specific sequence for assaying circulating FAP, demonstrating a sequence (VsPSQG) with specificity over PREP [21].

It is amongst the objects of the present disclosure to provide a specific probe for FAP that is specific over other related enzymes, resistant to degradation in areas of active inflammation and compatible with microscopy platforms.

### SUMMARY

The present disclosure is based in part on the development of peptide constructs, which are specifically cleavable by FAP and which upon cleavage generate a detectable signal, such as by an increase in signal, change in one or more physicochemical properties or increase in signal to noise ratio. The specificity of the disclosed constructs for FAP provides means of detecting FAP activity or expression and overcomes the disadvantages of existing constructs targeting FAP, which may not be sufficiently selective.

In a first aspect, there is provided a peptide construct that is selectively cleavable by FAP for use in a method of detecting FAP, the construct comprising a FAP cleavable peptide attached, conjugated or bound to one or more detectable moieties, wherein the construct cleavable by FAP comprises of the sequence:

(SEQ ID NO: 1)
A₋₂-A₋₁-A₀-A₊₁-A₊₂

wherein A₋₂ is a D- or L-amino acid or derivative thereof; A₋₁ is a D-amino acid, (β)Ala or derivative thereof; A₀ is Pro or derivative thereof; A₊₁ is Asn or derivative thereof; A₊₂ is a D- or L-amino acid or derivative thereof.

In one embodiment, A₋₂ may be selected from Val, Lys or derivative thereof; A₋₁ may be selected from (D)Ala, (D)Ser, (D)Thr, (β)Ala or derivative thereof; A₀ is Pro, (D)Pro or derivative thereof; A₊₁ is Asn, (D)Asn or derivative thereof; A₊₂ may be selected from Gln, Glu, Lys or derivative thereof.

In another embodiment, the peptide construct that is specifically cleaved by FAP may comprise of the sequence:

(SEQ ID NO: 2)
A₋₃−A₋₂-A₋₁-A₀-A₊₁-A₊₂-A₊₃-A₊₄

wherein A₋₂, A₋₁, A₀, A₊₁ and A₊₂ are as defined in the first aspect; A₋₃, A₊₃ and/or A₊₄ may be individually present or absent in the peptide sequence. When present, A₋₃, A₊₃ or A₊₄ may be a D- or L-amino acid or derivative thereof.

In an alternative embodiment, when A₋₃, A₊₃ and/or A₊₄ are present, A₋₃ is selected from Lys, Arg, Gln or Asn; A₊₃ is selected from Gly or Cys; and A₊₄ is selected from Lys, Arg, Gln or Asn.

In certain embodiments, A₋₂ is Val, A₋₁ is (D)Ser, A₀ is Pro, A₊₁ is Asn and/or A₊₂ is Gln.

In certain embodiments, A₋₃ is Lys, A₋₂ is Val, A₋₁ is (D)Ser, A₀ is Pro, A₊₁ is Asn, A₊₂ is Gln, A₊₃ is Gly and/or A₊₄ is Lys.

In one embodiment, the construct comprises the sequence Val-(D)Ser-Pro-Asn-Gln (SEQ ID NO: 3).

In one embodiment, the construct comprises the sequence Lys-Val-(D)Ser-Pro-Asn-Gln-Gly-Lys (SEQ ID NO: 4).

The peptide constructs as described herein, are typically less than 50, 25, 15, or 10 amino acids in length. In some embodiments, the peptide constructs comprise only 5-8 amino acids.

FAP exerts endopeptidase activity on the peptide constructs defined herein and preferentially cleaves the carboxy-terminal of the A₀/proline residue. The peptide construct and derivatives thereof disclosed herein are not preferentially cleaved by prolyl endopeptidase (also known as prolyl oligopeptidase or post-proline cleaving enzyme) nor dipeptidyl peptidases. Thus, the peptide constructs of the present disclosure are selective for FAP, particularly with respect to PREP and DPPs (e.g., DPP-4, DPP-8 and DPP-9). Importantly, the constructs disclosed herein are resistant to non-specific degradation in areas of active inflammation and demonstrate stability in molecules generated by activated immune cells, such as enzymes, reactive oxygen species and other inflammatory mediators.

Throughout this disclosure, three letter code is used to define the amino acids. For clarity, the three letter code for each amino acid are as the following: alanine (Ala), arginine (Arg), asparagine (Asn), aspartic acid (Asp), cysteine (Cys), glutamic acid (Glu), glutamine (Gln), glycine (Gly), histidine (His), isoleucine (lie), leucine (Leu), lysine (Lys), methionine (Met), phenylalanine (Phe), proline (Pro), serine (Ser), threonine (Thr), tryptophan (Trp), tyrosine (Tyr) and valine (Val). Naturally occurring peptides or proteins comprise of amino acids in the L-configuration. Unless amino acids are specified to be in the D-configuration (e.g., presented in the form of D-serine or ((D)Ser)), all amino acids refer to the L-configuration throughout this disclosure.

The term “derivative” as used herein refers to any modification(s) made to the molecule, such as modifications made to amino acids. The peptide construct of this disclosure may comprise one or more modified amino acids. Examples of modifications include, but not limited to, phosphorylation, methylation, acetylation, adenylylation, alkylation, amidation, cyclisation, deamidation, glycosylation, hydroxylation, halogenation, fluorination, isomerisation, sulfation, succinylation, myristoylation, oxidation, nitrosylation, biotinylation, SUMOylation, ubiquitination and pegylation.

In some instances, the peptide sequence may contain one or more beta-amino acids. The peptide sequence of the construct may also comprise one or more unnatural amino acids, such as citrulline, hydroxyproline, norleucine, 3-nitrotyrosine, nitroarginine or ornithine. Other modifications of the peptide sequence may include carbohydrate or lipid moieties, such as sugars or fatty acids, covalently linked to a side chain of one or more amino acids.

The FAP specific peptide construct may comprise further groups or molecules, typically attached to the N- or C-terminus of the peptide sequence. In one embodiment, the further groups or molecules are attached to the C-terminus of the peptide sequence. The further groups or molecules may include one or more amino acids, which may also further comprise one of more modifications such as being glycosylated or phosphorylated. In another instance, the one or more amino acids may comprise one or more D-enantiomer amino acids.

In addition, the peptide construct of this disclosure may be modified to enhance solubility and/or half-life upon being administered. For example, polyethylene glycol (PEG) and related polymers have been used to enhance solubility and/or the half-life of protein therapeutics in the blood. Accordingly, the peptide constructs of this disclosure may be modified, such as via the C-terminus, by PEG polymers and the like. PEG or PEG polymers means a residue containing poly(ethylene glycol) as an essential part. Such a PEG/PEG polymer may contain one or more further chemical groups which are necessary for the activity of the peptide constructs of this disclosure; which results from the chemical synthesis of the molecule; or which serves as a spacer to provide for optimal distance between the FAP cleavable peptide sequence and the PEG or other suitable molecule. In addition, such a PEG/PEG polymer may be formed of one or more PEG side-chains which are linked together. PEG groups with more than one PEG chain are called multiarmed or branched PEGs and may be suitable for use herein.

Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. Branched PEGs usually have 2 to 8 arms and are described in, for example, U.S. Pat. No. 5,932,462. Especially preferred are PEGs with two PEG side-chains (PEG2) linked via the primary amino groups of a lysine (Monfardini, C, et al., Bioconjugate Chem. 6 (1995) 62-69). The term “PEG” is used broadly to encompass any polyethylene glycol molecule, which comprises a number of repeating ethylene glycol (EG) units.

In an alternative embodiment, the peptide construct described herein may be attached to a non-PEG solubilising moiety, such as peptides containing multiple units of charged amino acids (e.g., polylysine). In another embodiment, the peptide may not be further modified to comprise any solubilising moiety.

The constructs described herein comprise at least one detectable moiety, such as a fluorescent moiety, which may be conjugated to the peptide by any suitable method. Typically, the detectable moiety will be conjugated to the peptide by way of a covalent bond. For example, the detectable moiety may be conjugated to the peptide by way of an amide bond. The detectable moiety may be conjugated anywhere along the peptide sequence, but in one embodiment, the detectable moiety is covalently bonded to the N or C terminal amino acid of the peptide, such as by way of an amide bond. In one embodiment, the signal-generating moiety is covalently bonded to the N terminal amino acid. Conjugation/covalent attachment via amino acid side chains, is also envisaged. The signal generating moiety may be directly conjugated/covalently bonded to the peptide or through a linker molecule between the detectable moiety and the peptide.

The constructs may further comprise a quencher moiety, which is designed to quench a signal, such as a fluorescent signal from the at least one detectable moiety, prior to peptide cleavage. Following peptide cleavage, the detectable moiety and quencher molecule are no longer in immediate proximity and the signal from the detectable moiety is no longer quenched. When employed, the quencher moiety may be conjugated or bound to the peptide in a similar manner to the detectable moiety as described above. However, the quencher moiety is not generally conjugated or bound to the same location on the peptide as the detectable moiety. In one embodiment, the detectable moiety is conjugated or covalently bound to the N or C terminal amino acid, with the quencher moiety conjugated or covalently bound via the C or N terminal amino acid respectively. The detectable moiety, fluorescent moiety and/or quencher moiety may be indirectly conjugated, for example via a suitable linker molecule, such as alkyl,alkylene, aromatic or heterocyclic (e.g., piperazine-type linkers) linkers, to the peptide.

In accordance with one teaching of the disclosure, the signal moiety may be a fluorescent moiety, which is initially quenched by the use of a quencher and the method of fluorescence resonance energy transfer (FRET) employed. Fluorescent signal detection may be an increase in fluorescence intensity, or an alteration in fluorescence lifetime, for example. Fluorescence may be detected, for example by, fluorescence spectroscopy, fluorescence microscopy, confocal fluorescence microscopy, fluorescence lifetime imaging microscopy, fluorescence image analysis, flow cytometry, laser scanning cytometry, plate multi-well fluorescence reader, or a scintillation counter.

The principle of FRET, also known as Förster resonance energy transfer (FRET), resonance energy transfer (RET), and electronic energy transfer (EET), is based on the transfer of energy from an excited donor dye to an acceptor dye or quenchers that are located in spatial proximity. Dark acceptors or quenchers are substances that absorb excitation energy from a fluorophore and dissipate the energy as heat; while fluorescent acceptors or quenchers re-emit much of this energy as light. Depending on the fluorescence quantum efficiency of the acceptor species, the energy transferred from the donor species to the acceptor species can either undergo non-radiative relaxation by means of internal conversion thereby leading to quenching of the donor energy, or can be emitted by means of fluorescence of the acceptor species.

FRET occurs between the electronic excited states of the donor species and acceptor species when they are in sufficient proximity to each other, in which the excited-state energy of the donor species is transferred to the acceptor species. The result is a decrease in the lifetime and a quenching of fluorescence of the donor species. In one application of this principle, as applicable herein, a fluorescent moiety is positioned to be in close proximity to a quencher moiety. In this configuration, the energy from the excited donor fluorescent moiety is transferred to the acceptor quencher moiety and dissipated as heat rather than fluorescence. However, when the fluorescent moiety is not in close proximity to the quencher moiety, such as following cleavage of the peptide by FAP, fluorescence of the fluorescent moiety is no longer quenched by the quencher moiety and can be detected by an increase in fluorescence intensity.

Fluorescent moieties suitable for use in the present invention can include a single molecule or molecular dye. Dyes useful for this invention include fluorescent, hydrophobic dyes that fluoresce in a range from 400 to 1000 nm. Classes of dyes include, but are not necessarily limited to oxonol, pyrylium, Squaric, croconic, rodizonic, polyazaindacenes or coumarins, scintillation dyes (usually oxazoles, benzothiadiazoles and oxadiazoles), aryl- and heteroarylsubstituted polyolefins (C2-C8 olefin portion), merocyanines, Rhodamines, sulphocyanines, carbocyanines, phthalocyanines, oxazines, carbostyryl, porphyrin dyes, dipyrrometheneboron difluoride dyes, aza-dipyrrometheneboron difluoride dyes, and oxazine dyes. Commercially available fluorescent dyes may be obtained from Thermofisher, Sigma Aldrich, for example.

Exemplary quencher dye compounds suitable for use in the present disclosure can include a single molecule or molecular dye. Suitable quencher dyes include e.g. DABCYL and QSYseries from Molecular Probes (www.probes.com), Dark Cy-dyes from Amersham Biosciences (www.amershambiosciences.com), Eclipse Dark Quencher dyes from Epoch Biosciences (www.epochbio.com), Black Hole Quencher dyes from Biosearch Technologies (www.biosearchtech.com), DYQ-dyes from Dyomics (www.dyomics.com), Black Berry Quenchers from Berry & Associates (www.berryassoc.com), QXL quenchers from AnaSpec, Inc. and ElleQuencherfrom Oswel (www.oswel.com). Additional quenchers include Methyl Red, Iowa Black FQ, Iowa Black RQ (Integrated DNA Technologies), IRDye QC-1 (Licor) and Si-Rhodamine-based NIR dark quenchers (Mycohin et al., J. Am. Chem. Soc. 2015, 137, 14, 4759-4765). Other fluorophore pairs commonly used in the field for FRET-base assays can be applied, such as cyan fluorescent protein (CFP) and yellow fluorescent protein (YFP). The fluorescent moieties and quencher dyes need to be sufficiently close when bound to the peptide, in order to ensure suitable quenching of any fluorescent signal, before peptide cleavage. The skilled addressee can easily test the quenching ability for longer peptides and whether or not the fluorescent and/or quencher moieties can be bound to the peptide ends, or if the fluorescent and/or quencher moieties may have to be conjugated or bound via an internal group of the peptide.

For example, in one teaching, the fluorescent moiety and quencher moiety pair may be MethylRed and 5-FAM directly conjugated and/or bound to the peptide construct. In one embodiment, the peptide construct is the probe FAP3, as defined herein.

In another embodiment, an alternative detectable moiety or a combination of detectable moieties may be attached to the peptide construct, such as luminescent molecule, fluorescent molecule, pH sensitive molecule or oxygen sensitive molecule. The optical signal that is generated by the peptide construct subsequent to cleavage by FAP can be used to infer FAP activity.

The signal generated from the detectable moiety may be detected using any suitable means. In one embodiment, the signal is an optical, such as a fluorescent signal. Optical signals can be detected by a variety of methods known in the art, including visually, spectrophotometrically and the use of optical sensors, including CCD and CMOS sensors, photodiodes and the like. Any suitable optical detection method may be employed and this should not be seen as limiting.

FAP expression is typically upregulated in diseased states or in solid organ malignancies by activated fibroblasts and high levels of FAP may be correlated with poor prognosis. Thus, the peptide constructs of the present disclosure may be applied to detect a level of FAP activity in cancers, such as lung cancer, pancreatic cancer, skin cancer, prostate cancer, brain cancer, colorectal cancer, head and neck cancer and breast cancer. In certain embodiments, the peptide construct may be used to detect a level of FAP in oral squamous cell carcinoma, ovarian cancer, pancreatic ductal adenocarcinoma and/or non-small cell lung cancer. In another instance, the peptide construct may be used to detect a level of FAP in chronic inflammatory conditions or fibrotic disease, such as hepatic fibrosis, idiopathic pulmonary fibrosis, myocardial infarction and rheumatoid arthritis. The applications of the peptide construct described herein should not be seen as limited to the conditions described above and may be applied to other diseased states associated with activated fibroblasts or altered, such as high FAP levels.

Importantly, the peptide construct and derivatives thereof disclosed herein are typically resistant to degradation at sites of inflammation and are not substantially cleaved by enzymes produced by immune cells, such as neutrophils. Further, the peptide construct and derivatives thereof are generally resistant to degradation by matrix remodelling enzymes. These enzymes may be, but are not limited to, matrix-degrading plasminogen activators or metalloproteinases (e.g., MMP-2, MMP-9, MMP-12 and MMP-13).

The peptide constructs described herein may be provided in a solution, or may be bound to a substrate, such as in the well of a micro titre plate, surface of a microfluidic channel, surface within a lateral flow system or any other suitable surface employed in analyte detection assays.

The constructs of the present disclosure may be further used in methods to detect a level of FAP expression or activity in a cell, a population of cells or bodily fluid in vitro, ex vivo or in vivo. These methods may comprise providing a tissue sample or a fluid sample from a subject, contacting a construct as defined herein with the sample and detecting any level of FAP expression or activity by detection of the detectable signal, e.g. optical signal, such as fluorescence, generated by cleavage of the peptide construct by FAP. The constructs of the present invention may be further used in methods to detect any effect (such as a therapeutic or cytotoxic effect) of a pharmaceutical agent on a population of cells in vitro, ex vivo or in vivo.

The present disclosure also extends to a kit for use in the detection or quantitation of a level of FAP expression or activity, the kit comprising a peptide construct attached, conjugated or bound to one or more detectable moieties, as described herein, together with instructions describing how the peptide construct may be used to detect a level of FAP expression or activity in a sample.

The kit may further comprise one or more of the following: a sample collection device; suitable buffers for collecting the sample in; a positive control to confirm that a suitable sample has been provided; a negative control, in order to provide a baseline of activity.

### DETAILED DESCRIPTION

The present disclosure is further described by way of example with reference to Figures, which show:

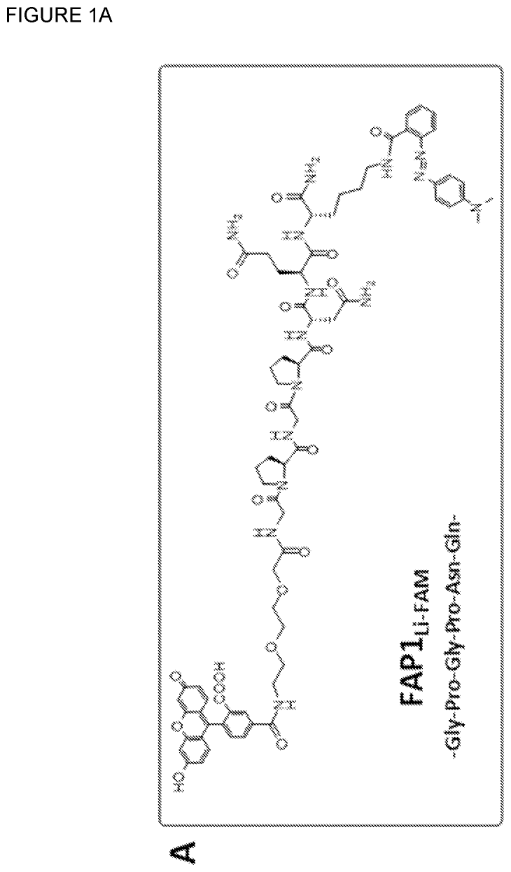

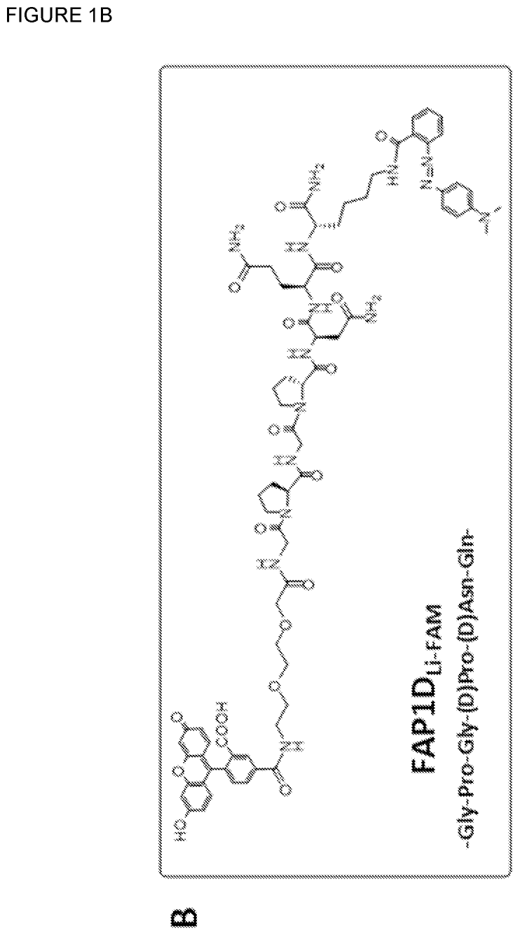

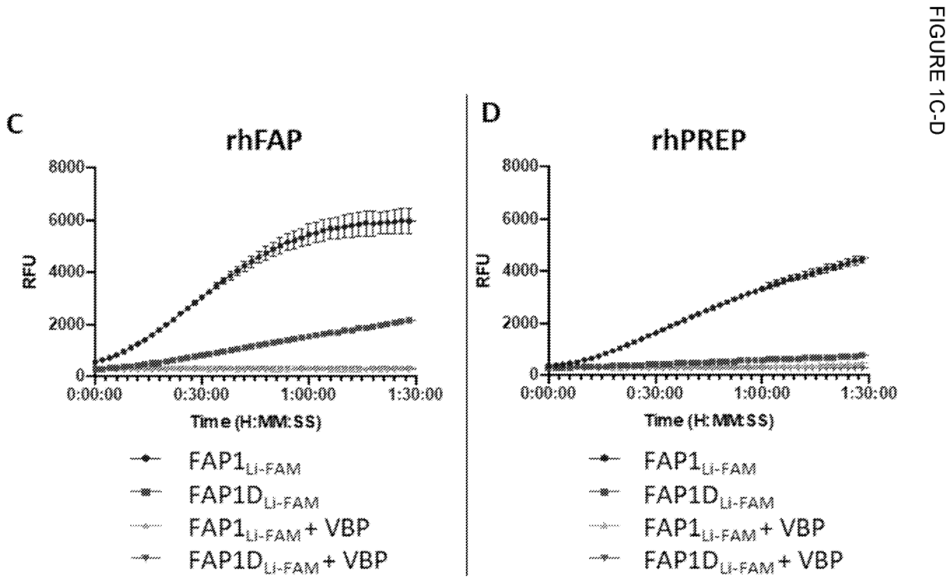

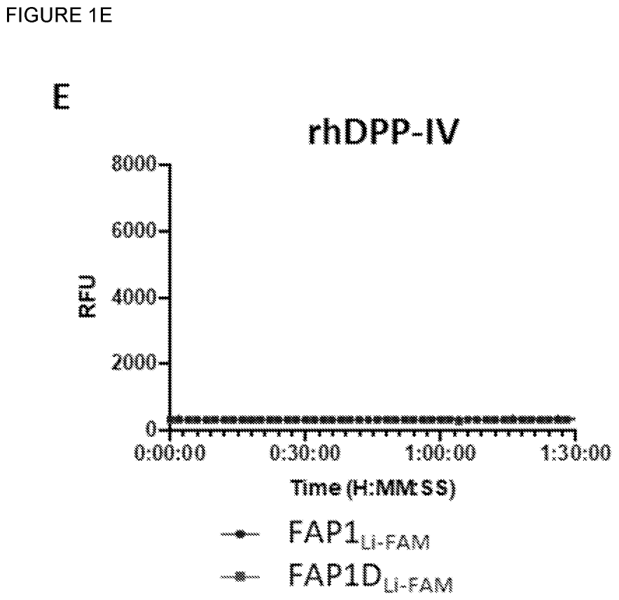

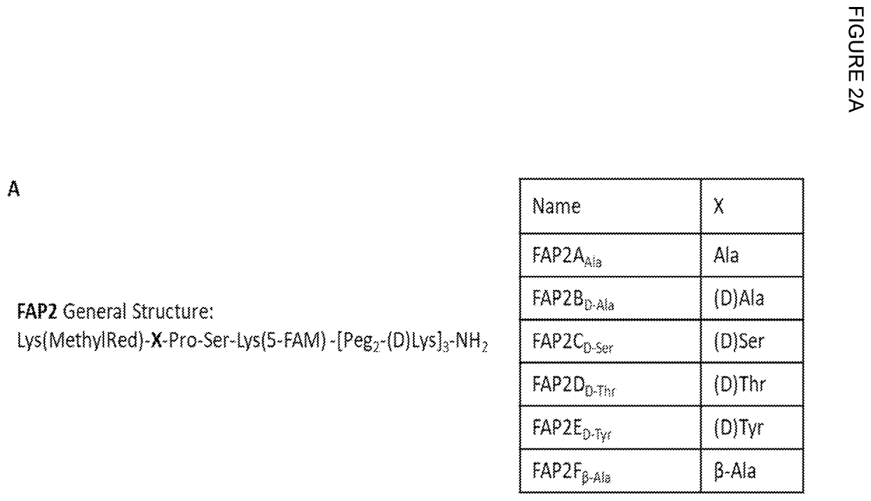

FIG. 1: FAP1 probes on recombinant human enzymes rhFAP, rhPREP and rhDPPIV to assess endo and exopeptidase activity. (A) The structure of FAP1_{Li-FAM}; (B) The structure of FAP1D_{Li-FAM}; (C) FAP1_{Li-FAM} is cleaved rapidly by rhFAP, FAP1D_{Li-FAM} shows lower rate of cleavage and ValBoro-Pro inhibits both probes; (D) rhPREP cleaves FAP1_{Li-FAM} and FAP1D_{Li-FAM}, although FAP1_{Li-FAM} is cleaved more rapidly. ValBoro-Pro inhibits cleavage of both probes by rhPREP; (E) Neither FAP1_{Li-FAM} or FAP1 D_{Li-FAM} are cleaved by rhDPPIV. Representative plots shown, n=3 for each experiment, run in triplicate.

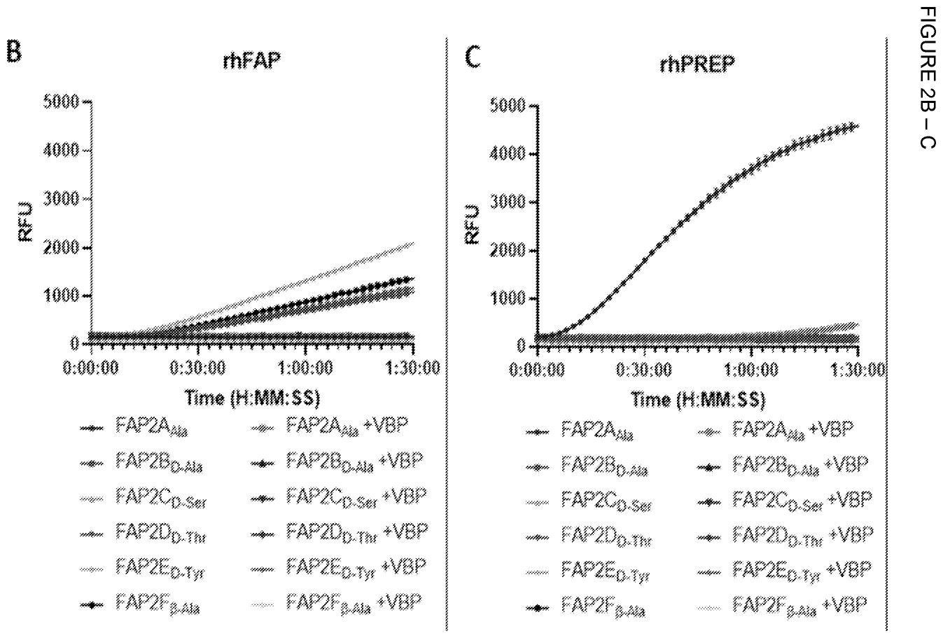

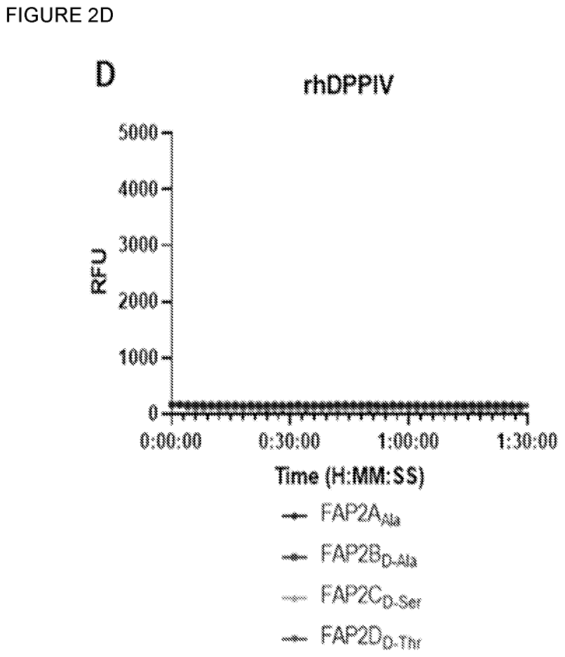

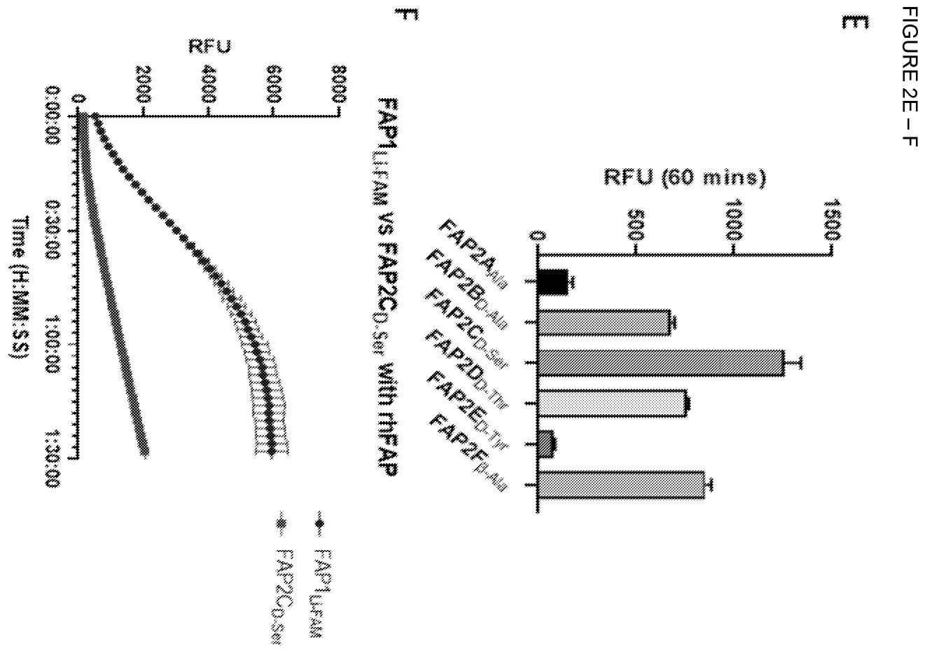

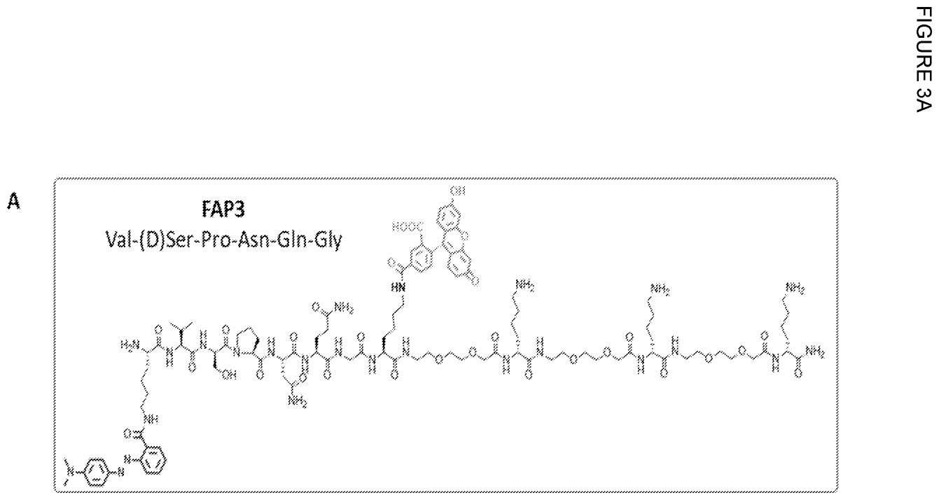

FIG. 2: Iterations of probe FAP2 to determine a sequence specific for FAP over PREP. (A) The sequence of FAP2 and a table showing the amino acid in the X position for each iteration; (B) Assessment of the probes against rhFAP showing relative intensity and ValBoro-Pro inhibiting cleavage. FAP2C_{D-Ser} shows highest rate of cleavage; (C) Assessment of the probes against rhPREP showing iteration FAP2A_{Ala} is cleaved by rhPREP; (D) Assessment of the probes against DPPIV showing none of the iterations are cleaved; (E) Comparison of the relative fluorescence intensity of each probe iteration at 60 minutes; (F) Comparison of FAP1_{Li-FAM} with FAP2C_{D-Ser} showing the signal intensity of FAP1 is higher within 90 minutes. Representative images, n=3, mean RFU plotted in bar graph and error bars show standard deviation.

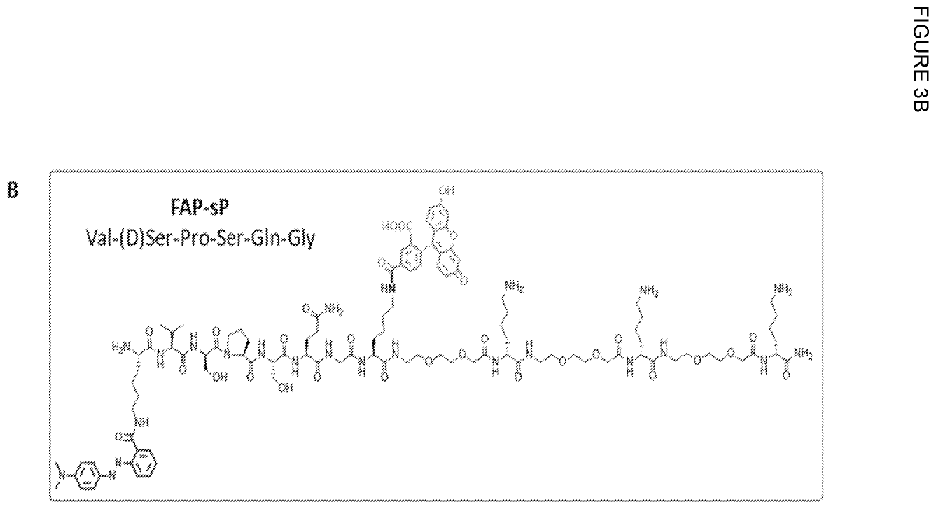

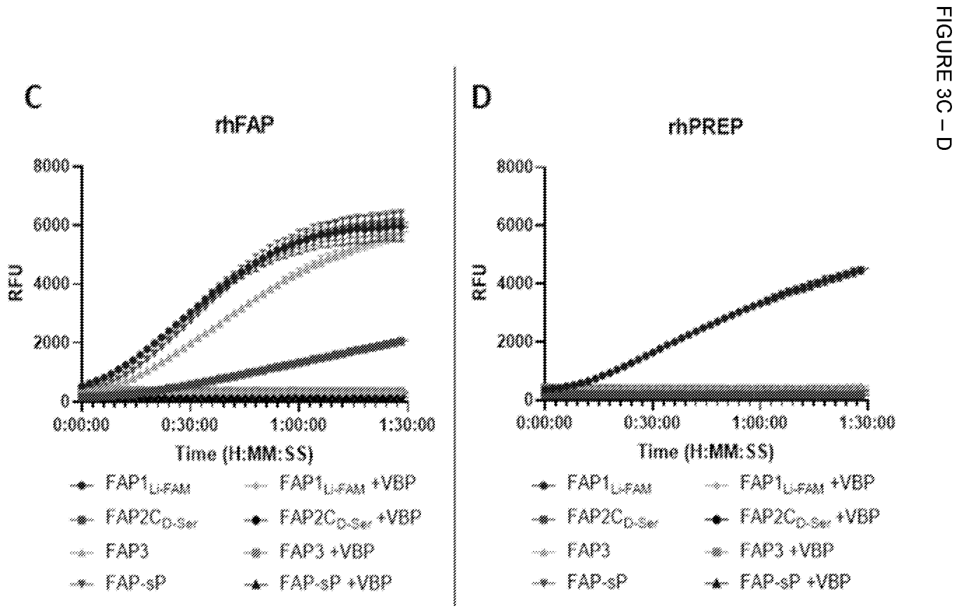

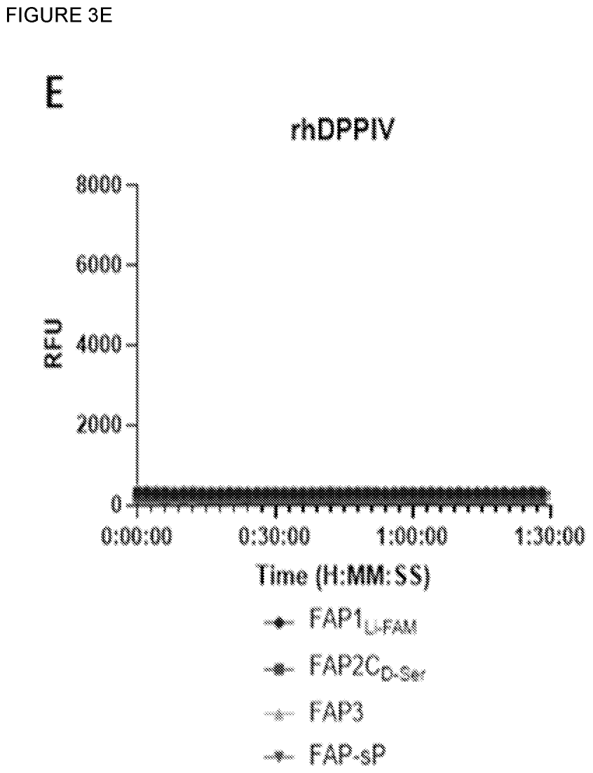

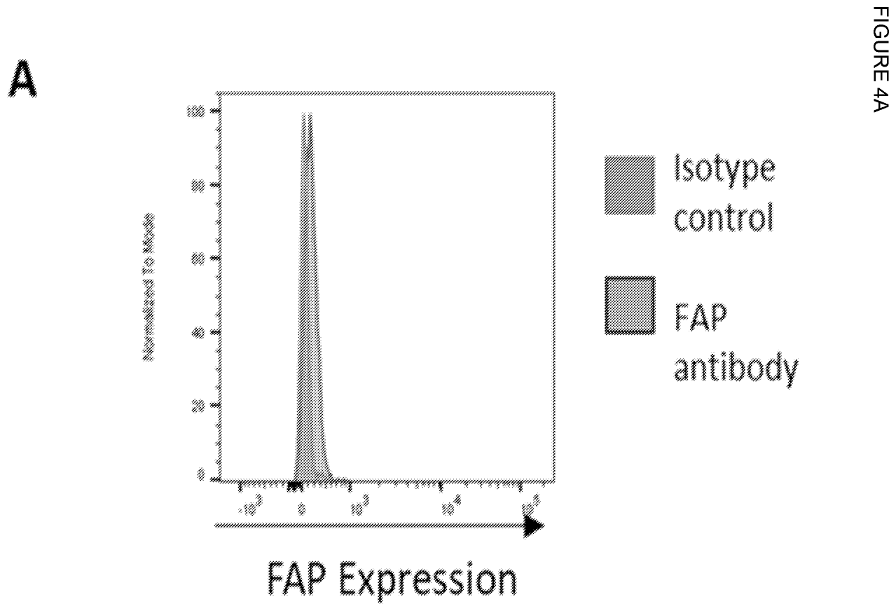

FIG. 3: FAP3 assessment against FAP-sP demonstrates FAP specificity. (A) The structure of FAP3; (B) The structure of FAP3-sP; (C) Assessment of FAP-3 and FAP-sp when compared to prior iterations; (D) rhPREP assessment demonstrating no rhPREP cleavage for FAP3 or FAP-sp; (E) DPPIV assessment demonstrating no exopeptidase activity. Representative plots, n=3 for each experiment run in triplicate.

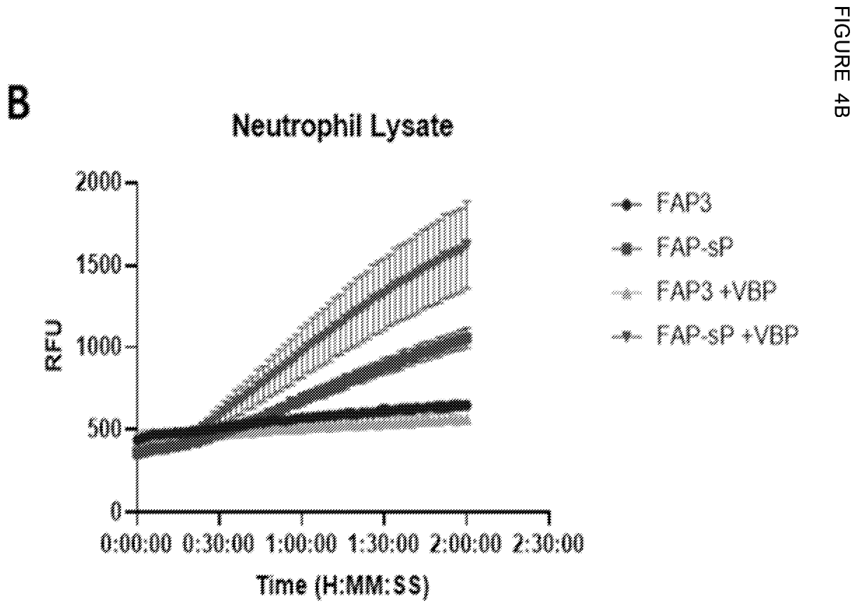

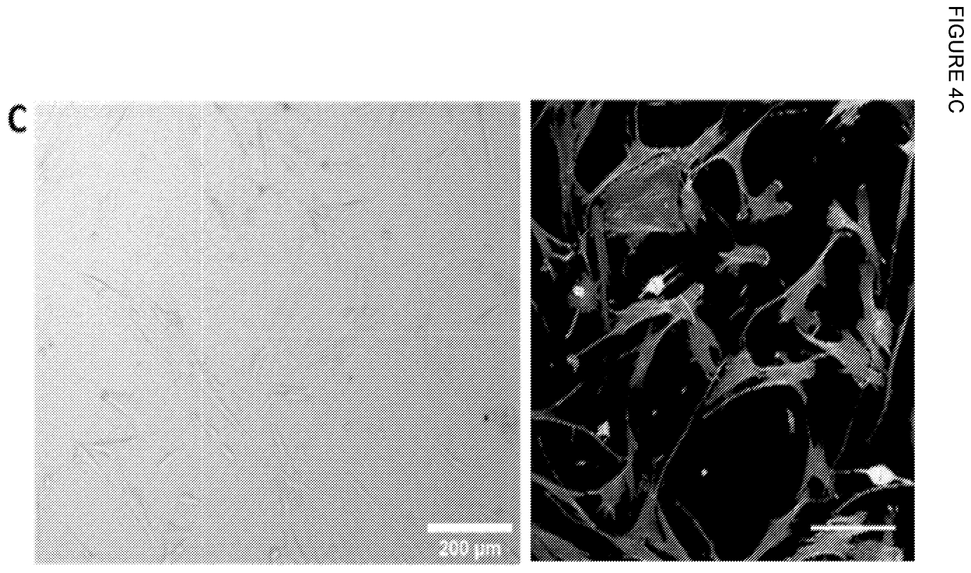

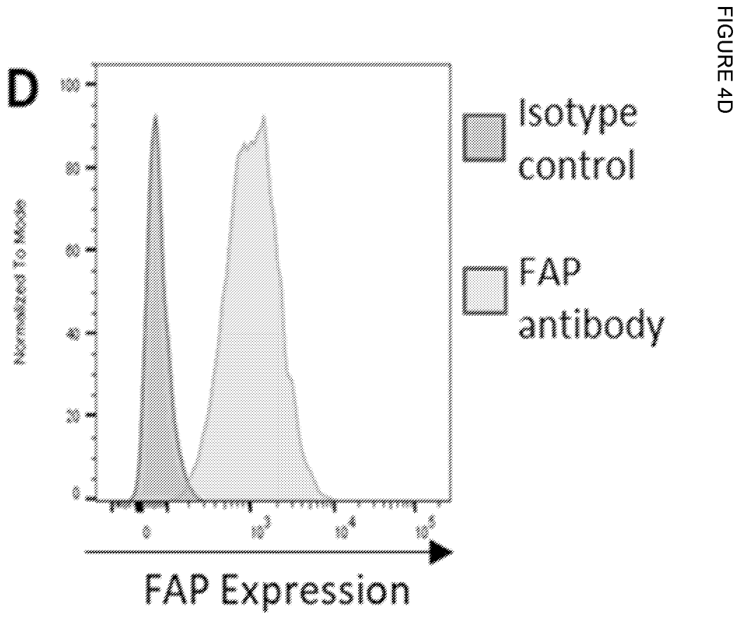

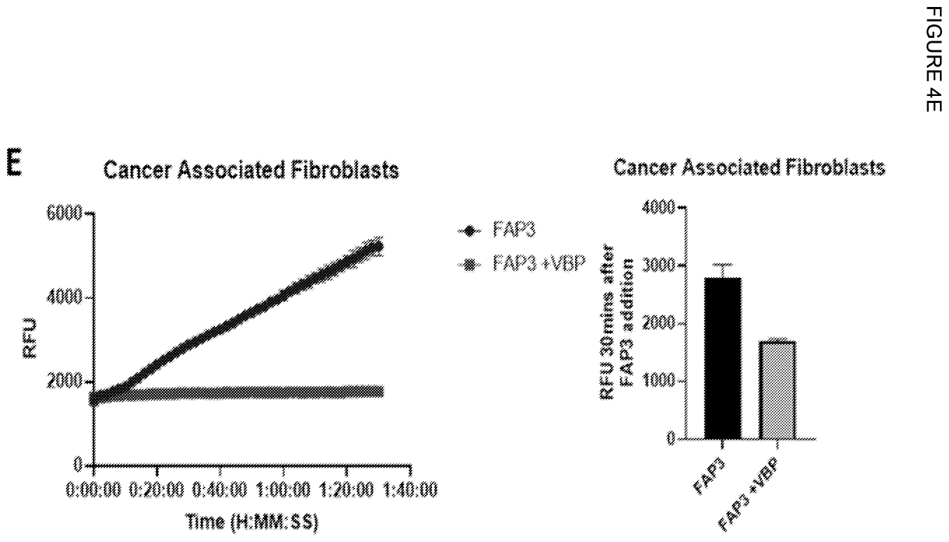

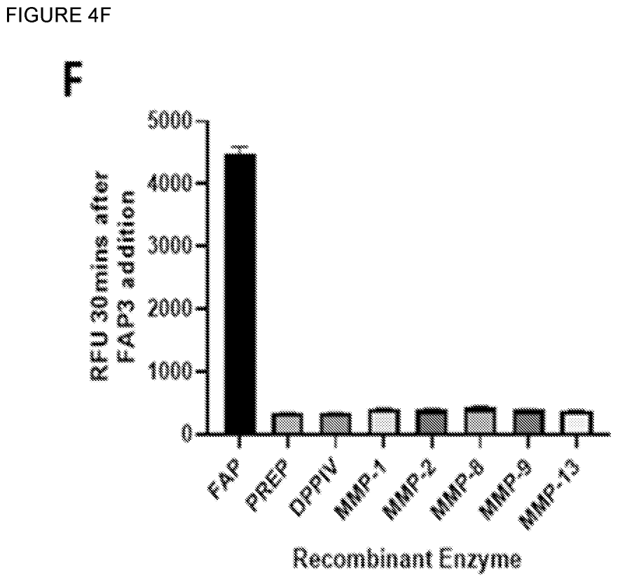

FIG. 4: FAP3 and FAP-sP probes assessed on activated neutrophil lysate and cancer associated fibroblasts (CAFs). (A) Flow cytometry confirmed that neutrophils were FAP negative; (B) Assessment in activated neutrophil lysate revealed FAP-sP was cleaved but FAP3 was not, and FAP-sP was cleaved in the presence of VBP; (C) Cancer associated fibroblasts in culture (brightfield, 200 μm scale bar) and stained for FAP (red), DAPI (blue) and phalloidin (green), scale bar 100 μm; (D) Flow cytometry confirmed CAFs are FAP positive; (E) When incubated with CAFs FAP3 was cleaved and signal was abrogated in the presence of VBP; (F) FAP3 demonstrates no activity for other recombinant enzymes (MMPs) likely to be present in NSCLC. n=2 for neutrophil lysate testing and MMP testing, n=3 for CAF testing, each experiment run in triplicate. Representative images shown.

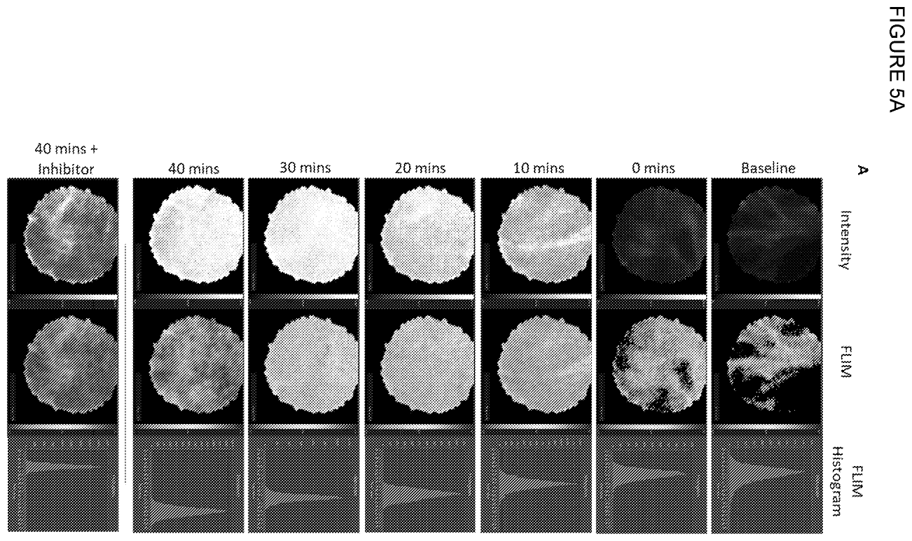

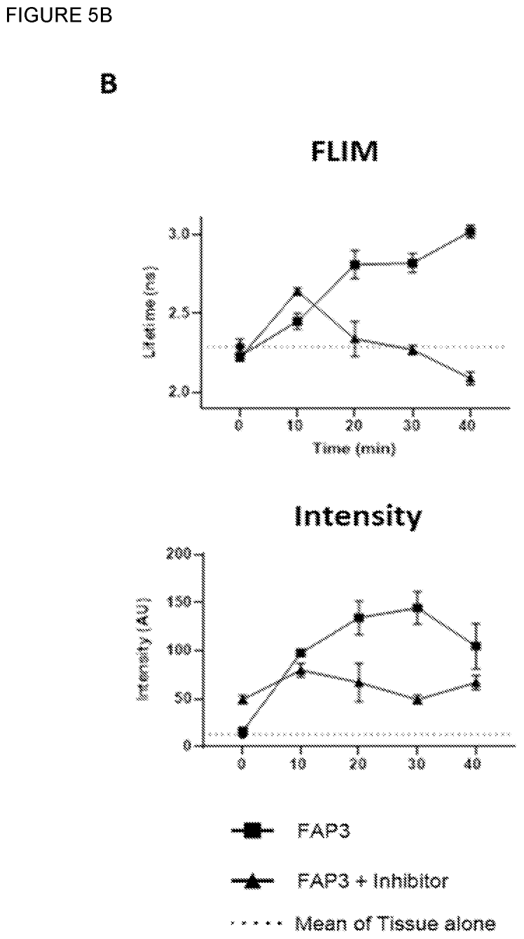

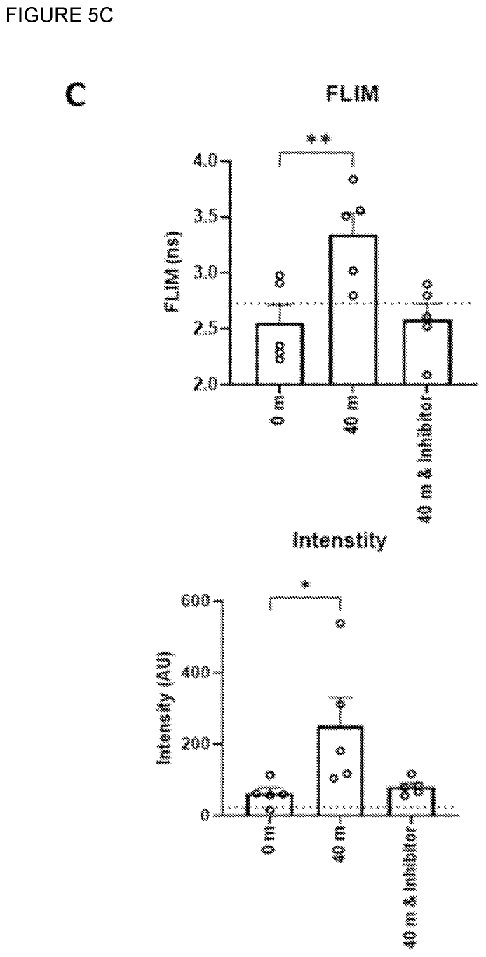

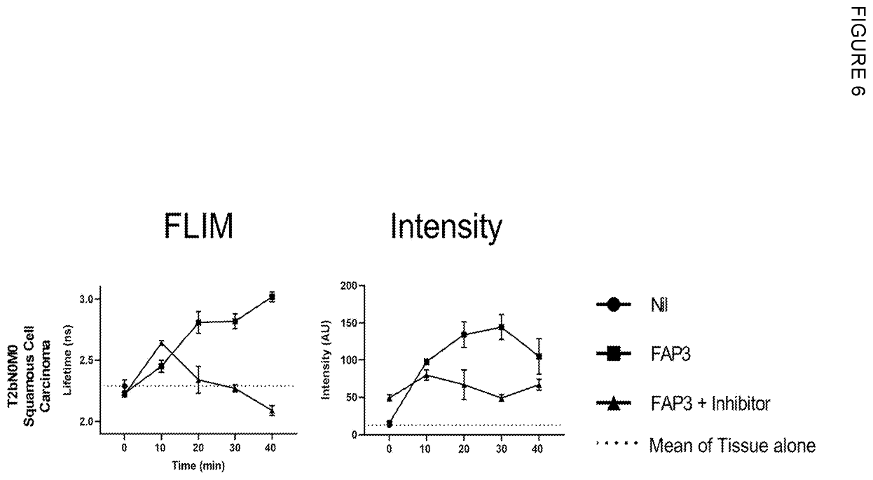

FIG. 5: Fluorescence lifetime imaging in NSCLC tissue using FAP3 probe and a clinically tractable FLIM system. (A) Representative images showing the change in intensity and fluorescence lifetime of a NSCLC tumour tissue sample over time; (B) Representative plots showing the change in intensity and fluorescence lifetime over 40 mins on a NSCLC tissue sample; (C) Aggregate analysis across 5 NSCLC samples at 40 minutes demonstrating a significant increase in fluorescence lifetime and intensity, with inhibition in the presence of VBP. N=5, analysis by paired t-test, *=p<0.05, **=p<0.01.

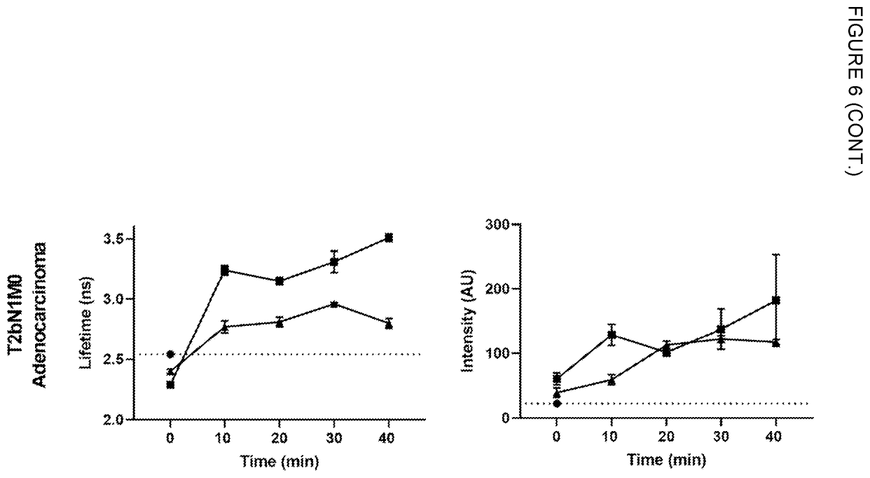

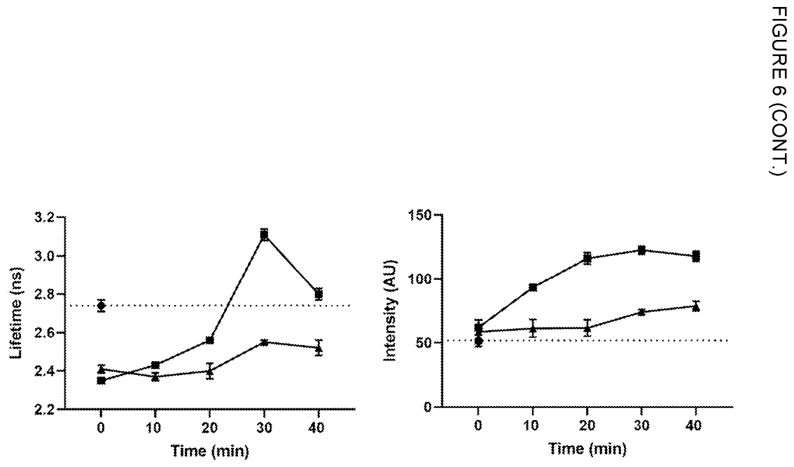

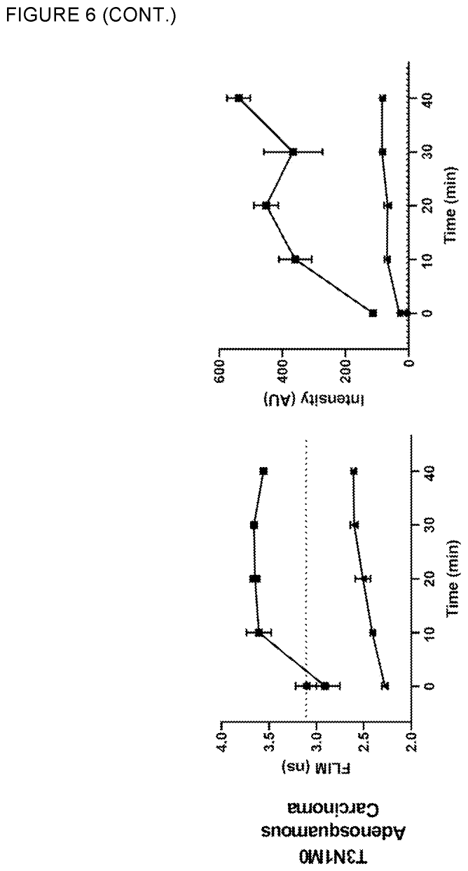

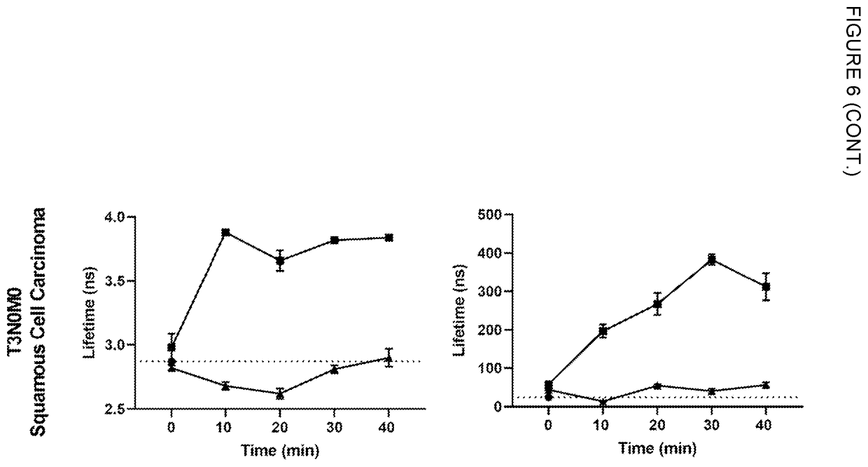

FIG. 6: The lifetime and FLIM measurements of 5 patient samples. Histological stage and subtype demonstrated on the y-axis and measurements of intensity and lifetime for each sample shown over time, with and without added inhibitor. Dotted line demonstrates the intrinsic values for each sample.

### METHODS

### Chemical Synthesis

Commercially available reagents were used without further purification. Methyl Red (MR) was purchased from Sigma, 5-Carboxyfluorescein (5-FAM) was purchased from Carbosynth. Ltd., [2-[2-(Fmoc-amino)ethoxy]ethoxy]acetic acid was purchased from Iris Biotech. Analytical reverse-phase high-performance liquid chromatography (RP-HPLC) was performed on an Agilent 1100 system equipped with a Kinetex XB-C18 reverse-phase column (50×4.6 mm, 5 μm) with a flow rate of 1.0 mL/min and eluting with H₂O/CH₃CN/HCOOH (95/5/0.1) to H₂O/CH₃CN/HCOOH (5/95/0.1) over 6 min, holding at 95% CH₃CN for 2 min, with detection at 254 nm and 495 nm and by evaporative light scattering. Semi-preparative RP-HPLC was performed on an Agilent 1100 system equipped with a Zorbax Eclipse XDB-C18 reverse-phase column (250×9.4 mm, 5 μm) with a flow rate of 2.0 mL/min and eluting with 0.1% HCOOH in H₂O (A) and 0.1% HCOOH in CH₃CN (B), with a gradient of 5 to 95% B over 30 min and additional isocratic period of 5 min. Electrospray ionization mass spectrometry (ESI-MS) analyses were carried out on an Agilent Technologies LC/MSD Series 1100 quadrupole mass spectrometer (QMS) in ESI mode. MALDI spectra were acquired on a Bruker Ultraflextreme MALDI-TOF MS with a matrix solution of sinapic acid (10 mg/mL) in H₂O/CH₃CN/TFA (50/50/0.1).

### General Solid-Phase Synthesis Methods

Manual peptide synthesis was performed on aminomethyl-ChemMatrix resin using an Fmoc-protected Rink amide linker. General procedures were as follows:

### Coupling of Rink Amide Linker

The Fmoc-Rink linker (4-[(R,S)-a-[1-(9H-Fluoren-9-yl)-methoxy-formamido]-2,4-dimethoxybenzyl-phenoxyacetic acid) (0.54 g, 1.0 eq) was dissolved in DMF (10 mL) and Oxyma (0.14 g, 1.0 eq.) was added and the mixture was stirred for 10 min. Diisopropylcarbodiimide (DIC, 155 μL, 1.0 eq.) was then added and the solution stirred for 1 min before adding it to aminomethyl-ChemMatrix resin (1.0 g, 1.0 mmol/g). The resulting mixture was stirred at 50° C. for 45 min and washed with DMF (3×10 mL), DCM (3×10 mL) and MeOH (3×10 mL). Finally the resin was treated with Ac₂O:Py:DMF (2:3:15) for 30 min in order to cap any remaining free amino groups and was washed again with DMF (3×10 mL), DCM (3×10 mL) and MeOH (3×10 mL). Resin loading was calculated as ˜0.58 mmol/g via spectrophotometric test [22].
Fmoc deprotection: to the resin pre-swollen in DCM 20% piperidine in DMF was added and the mixture stirred for 2×10 min. The solution was drained and the resin washed with DMF (3×10 mL), DCM (3×10 mL) and MeOH (3×10 mL).
Amino acid coupling: A solution of the appropriate D or L-amino acid (3.0 eq per amine) and Oxyma (3.0 eq) in DMF (0.1 M) were stirred for 10 min. DIC (3.0 eq) was added and stirred for 1 min. The pre-activated mixture was then added to the resin pre-swollen in DCM and the reaction heated at 50° C. for 30 min. The solution was drained and washed with DMF (3×10 mL), DCM (3×10 mL) and MeOH (3×10 mL). The completion of the coupling reactions was monitored by Kaiser or Chloranil tests (when secondary amines are involved). Fmoc-Lys(Dde)-OH was used as an orthogonal reagent to allow introduction of the dyes. Fmoc-Lys(Methyl Red)-OH was used as building block to add the quencher.
Coupling of other carboxylic acids: Coupling of {2-[2-(Fmoc-amino)ethoxy]ethoxy}acetic acid (PEG) and 5-Carboxyfluorescein (5-FAM) was carried out following the same procedure as described for the amino acid couplings.
Dde deprotection in presence of Fmoc protecting group was achieved as previously reported: 1.25 g of NH₂OH·HCl and 0.918 g of imidazole were suspended in 5 mL of NMP and the mixture was sonicated until complete dissolution; 5 volumes of this solution were diluted with 1 volume of CH₂Cl₂ and added to the resin. After 3 h the solution was drained and the resin washed with DMF (3×10 mL), DCM (3×10 mL) and MeOH (3×10 mL).
Cleavage and purification: The resin (100 mg) pre-swollen in DCM was treated with a cleavage cocktail of TFA:triisopropylsilane(TIS):water (95:2.5:2.5) (2 mL) for 3 h. The reaction solution was drained and the resin washed with the cleavage cocktail (1 mL). The combined solution was precipitated against cold ether, centrifuged (×3) and purified by RP-HPLC on a C-18 semi-preparative column. The desired fractions containing the product were collected and lyophilized to afford the pure compounds.
   Assessment with Recombinant Enzymes

Assays were undertaken in triplicate in blackened 384 well plates on ice prior to spectral reads. All solutions were diluted in TRIS buffer (25 nM Tris, 250 nM NaCl, pH7.5). Probes were used at 5 μM and inhibitors (namely Val-boroPro (VBP), Merck) at 10 μM, unless otherwise stated. All wells contained a final volume of 20 μl to include buffers, inhibitors (if used), substrates and recombinant human enzymes (at a concentration of 0.1 μg/ml unless otherwise stated). Plates were sealed with an optical plate sealer (Biolegend) and were read in a preheated spectrophotometer (Synergy Biotek) at 380/460 nm for control substrates and 480/530 nm for FAP probes.

### Assessment on Neutrophil Lysate

Neutrophils were isolated from peripheral blood of healthy volunteers by discontinuous percoll gradients, as previously described [23]. Cells were resuspended in PBS (at 10 million cells/ml) and stimulated with 1 μM calcium ionophore, then lysed with 1% Triton-X-100. Lysate was used at 1:1 for experiments, replacing recombinant enzymes in the assay as described above, with PBS as the buffer.

### Assessment on Cancer Associated Fibroblasts (CAFs)

CAFs were isolated from NSCLC patient samples as previously described methods known in the art. Briefly, tissue samples were minced with forceps and incubated for an hour in prewarmed RPMI media (Gibco) containing collagenase IV [2 mg/ml](Sigma) and DNase [0.2 mg/ml](Sigma). Samples were spun at 350 g for 5 minutes and red blood cells were lysed from samples using RBC lysis buffer (BioLegend) in 10 mls for 10 minutes. Following a further spin at 350 g for 5 minutes, cells were seeded in culture flasks in DMEM containing 10% FCS, 1% Penicillin-Streptomycin, 1% L-Glutamine and 10% Insulin Transferrin Selenium (ITS). 24 hours after seeding, non-adherent cells were washed from the flasks. Cells were maintained by standard cell culture methods and by passage 2, the predominant cell type was CAFs, assessed by flow cytometry markers and morphology. CAFs between passage 4-9 were used to assess FAP probes and confirmed to be FAP^{hi} by flow cytometry.

CAFs were seeded into 96 well plates in a 100 μl media (complete DMEM as described above) at a density of 1×10⁵ cells/ml to form a confluent monolayer within 24 hours. Wells containing cells had media removed and then were washed before buffer (DPBS)+/− inhibitors were added with the imaging probes. Immediately after adding imaging probes, the plate was transferred to the prewarmed spectrophotometer as above and read at 480/530 nm for one hour.

For imaging, CAFs were seeded in glass bottom chambers (Ibidi), grown, and fixed with 4% paraformaldehyde for 20 minutes at 4° C. Following three washes, cells were permeabilised with 0.2% Triton X-100, quenched with ammonia chloride (50 mM) for 5 minutes and blocked with 1% BSA. FAP antibody at 1:100 (AF3715, R&D systems) was incubated overnight at 4° C., washed, incubated with secondary conjugated to Alexa Fluor 633 (1:1000) and Alexa Fluor 488 Phalloidin 5 μL/250 μL (A12379, Life Technologies) for 1 hour and finally incubated with DAPI (D1306, Life Technologies) in the dark for 5 minutes. Images were taken on Leica SP5 confocal microscope (Leica Microsystems, Wetzlar, Germany) using dedicated laser excitation at 405 nm, 488 nm and 633 nm.

### Flow Cytometry

Cells collected in suspension (CAFs or neutrophils) were stained with a live dead marker Zombie UV (1:1000, Biolegend) for 30 min at room temperature in DPBS (Gibco). Cells were then washed and stained with an anti-FAP-APC antibody (1:20, R&D Systems) for 20 mins at 4° C. in DPBS supplemented with 2% FCS. After washing cells were fixed in a 1:1 solution of fixation buffer (Biolegend) and DPBS with 2% FCS overnight at 4° C. before data acquisition on a LSR6Fortessa analyser (BD Biosciences). Flow cytometry data was then analysed using FlowJo version 10.7.1. Compensation was carried out using single stain control UltraComp eBeads (Invitrogen) and isotype control samples were stained using iso-anti-FAP-APC (1:20, R&D Systems). FAP expression was determined by gating on singlet, live cells and then looking at anti-FAP-APC signal compared to the isotype control.

### Fluorescence and Lifetime Imaging

For imaging solutions, varying concentrations of rhFAP were prepared in blackened eppendorfs and FAP3 to a final concentration of 5 μm added and imaged as below. For biological specimens, NSCLC patient tissue samples from surgical resection were used fresh or snap frozen and stored at −80 until required. Small fragments (approx. 4 mm³) incubated at 37° C. in a 96 well plate in phenyl-red free DMEM (Gibco) containing 10% FCS, 1% Penicillin-streptomycin and 1% L-Glutamine. For the inhibitor fragments VBP (1:1000) was added. Samples were then imaged using a clinically tractable novel fluorescence lifetime imaging system, KronoScan, with an imaging fibre providing a 400×400 um field of view. KronoScan incorporates a supercontinuum laser source, in this case tuned for excitation at 488 nm, with a confocal laser scanning system and a time-resolved spectrometer. This spectrometer contains a 512 channel single photon avalanche diode (SPAD) sensor, allowing for the rapid collection of time resolved spectral fluorescence lifetime data. Fluorescence intensity and lifetime images were collected at 160×160 pixels over a 498-570 nm spectral range with an exposure time of 13 μs per pixel. This led to an imaging rate of ˜3 frames per second.

For each condition the tumour samples were imaged using a fibre placed at the tip of the tissue (baseline), and then again at 10 minute intervals following the additions of equimolar concentrations of probe+/− Inhibitor. For imaging solutions, the fibre was held in the solution and imaged every 5 minutes. Each condition and time-point data for both fluorescence intensity and lifetime were collected. Imaging sequences had non-relevant frames removed, and the entire field of view was analysed. Analysis was undertaken using a bespoke software suite utilising the rapid lifetime determination (RLD) method. The RLD method utilises two-time bins for reduced data load and high speed analysis whist retaining reasonable lifetime approximation for single exponential decays. Whilst the sample analyses are likely multi-exponential in character the lightweight RLD algorithm provides a good approximation to the intensity weighted average lifetime observed. Data had background subtraction, lightfield normalisation and an intensity threshold (of 20) applied through all sequences. Intensity data are provided as relative units (RU) and lifetime as nanoseconds (ns).

### Examples

### Optimising Fragment for FAP Specificity

A previously published FAP optical reporter sequence was synthesised, which had been determined to be FAP specific over DPPIV but had not been assessed against PREP [20]. We synthesised the reported peptide sequence but for FRET pairing we utilised a Carboxyfluorescein (FAM) fluorophore with a Methyl-red quencher (FIG. 1A) as an alternative to Cy5.5/QSY21. Assessment of this probe (termed FAP1_{Li-FAM}) (Table 1) sequence against the recombinant enzymes FAP and PREP demonstrated endopeptidase activity through PREP as well as FAP (FIG. 1C-D), which was independently demonstrated by Bainbridge et al for the same sequence [21]. Confirming previous reports, DPP-IV did not cleave the probe (FIG. 1E) signifying absent exopeptidase activity as the proline residues are not in terminal positions. The broad-spectrum (FAP and PREP) inhibitor, Val-boroPro (VBP), successfully abrogated the signal (FIG. 1D).

The construct has Gly-Pro repeats susceptible to PREP cleavage, therefore modifications to block the endopeptidase action by the second proline were made with D-proline and D-asparagine to try and confer a uniquely FAP cleavable probe (structure shown in FIG. 1B). This demonstrated partial reduction in activity in the presence of PREP (FIG. 1D), but also demonstrated that FAP activity was significantly reduced (FIG. 1C). Further iterations were assessed against FAP1_{Li-FAM} and FAP1D_{Li-FAM} activity to assess cleavage efficacy.

Whilst there was an improvement in the specificity, FAP1 D_{Li-FAM} still demonstrated PREP cleavage so additional compounds were synthesised to try to overcome this. Shorter versions of the previous sequence were synthesised, and the amino acid prior to proline altered in six iterations (structures shown in FIG. 2A), as it had been previously reported that such modification can introduce FAP specificity [21]. These compounds were then assessed with recombinant human enzymes (FIG. 2B-D). Insertion of Ala (FAP2A_{Ala}) demonstrated cleavage by PREP and FAP activity was absent, and the insertion of D-Tyr also resulted in loss of FAP activity. The iterations containing D-Ala, D-Ser, D-Thr and β-Ala in the position prior to proline all demonstrate FAP specificity over PREP, however, with reduced signal that may preclude clinical translation. Assessment against DPP-IV confirmed no iterations had introduced exopeptidase activity (FIG. 2D). As the clinical setting requires rapid optical readout, further iterations were made using D-Ser (FAP2C_{D-Ser}) which demonstrates FAP specificity (FIG. 2B-C) without PREP cleavage whilst displaying the strongest signal compared to the other iterations (FIG. 2E). Comparison with the FAP1_{Li-FAM} compound demonstrated a low signal (FIG. 2F), and this served as a comparison to additional compounds.

TABLE 1
Summary showing amino acid chain used for each probe.

| | | SEQ ID |
|---|---|---|
| Probe | Sequence | NO. |
| FAP1_{Li-FAM} | FAM-Peg₂-Gly-Pro-Gly-Pro-Asn-Gln-Lys(MethylRed)-NH₂ | 5 |
| FAP1D_{Li-FAM} | FAM-Peg₂-Gly-Pro-Gly-(D)Pro-(D)Asn-Gln-Lys(MethylRed)-NH₂ | 6 |
| FAP2A_{Ala} | Lys(MethylRed)-Ala-Pro-Ser-Lys(5-FAM)-[Peg₂-(D)Lys]₃-NH₂ | 7 |
| FAP2B_{D-Ala} | Lys(MethylRed)-(D)Ala-Pro-Ser-Lys(5-FAM)-[Peg₂-(D)Lys]₃-NH₂ | 8 |
| FAP2C_{D-Ser} | Lys(MethylRed)-(D)Ser-Pro-Ser-Lys(5-FAM)-[Peg₂-(D)Lys]₃-NH₂ | 9 |
| FAP2D_{D-Thr} | Lys(MethylRed)-(D)Thr-Pro-Ser-Lys(5-FAM)-[Peg₂-(D)Lys]₃-NH₂ | 10 |
| FAP2E_{D-Tyr} | Lys(MethylRed)-D-Tyr-Pro-Ser-Lys(5-FAM)-[Peg₂-(D)Lys]₃-NH₂ | 11 |
| FAP2F_{β-Ala} | Lys(MethylRed)-(β)Ala-Pro-Ser-Lys(5-FAM)-[Peg₂-(D)Lys]₃-NH₂ | 12 |
| FAP3 | Lys(MethylRed)-Val-(D)Ser-Pro-Asn-Gln-Gly-Lys(5-FAM)-[Peg₂- | 13 |
| | (D)Lys]₃-NH₂ | |
| FAP-sP | Lys(MethylRed)-Val-(D)Ser-Pro-Ser-Gln-Gly-Lys(5-FAM)-[Peg₂- | 14 |
| | (D)Lys]₃-NH₂ | |

### Improvement of Signal-to-Noise

Using the D-Ser iteration we increased the peptide chain to include Asn to derive a novel sequence (FAP3: Lys(MethylRed)-Val-(D)Ser-Pro-Asn-Gln-Gly-Lys(5-FAM)-[PEG₂-(D)Lys]₃-NH₂) or with Ser (FAP-sP) and to act as a comparator (structures shown in FIG. 3A-B). FAP-sP was previously reported by Bainbridge to be utilised as a serum FAP detection probe [21]. Both sequences demonstrated FAP specificity (FIG. 3C) with improved signal characteristics which were further confirmed by MALDI-TOF analysis (data not shown). Testing also confirmed no cleavage by PREP or DPPIV for either compound (FIGS. 3D-E).

### Assessment of Probe in Biological Environments

To assess whether the sequence had sufficient robustness for clinical translation we assessed whether; i) the probe remains intact in areas of low FAP but high protease activity, and ii) the imaging probe can detect physiological levels of FAP. To ensure specificity is maintained in an inflamed environment the probe was assessed against activated neutrophils as these are one of the most predominant leucocyte cell subtypes in cancer [24]. Neutrophils were confirmed to be negative for FAP (FIG. 4A). FAP3 was stable in the presence of neutrophil lysate, however, FAP-sP was cleaved (FIG. 4B) in a non-FAP dependent manner. Subsequent analysis by MALDI-TOF revealed all FAP-sP probe was cleaved by activated neutrophil lysate, but FAP3 remained intact (data not shown).

Cancer associated fibroblasts were isolated and cultured from NSCLC patient samples (FIG. 4C) and confirmed to be FAP expressing (FIG. 4D). FAP3 was cleaved by FAP⁺CAFs, with inhibition of signal when co-incubated with Val-boroPro (FIG. 4E). Finally, to ensure stability in the presence of other matrix remodelling proteases found to be upregulated in NSCLC, FAP3 was assessed against a panel of MMP's including 2, 9, 13 demonstrating no activity (FIG. 4F). Therefore, FAP3 is a FAP specific sequence over PREP, DPP-IV and MMP's, that can detect FAP within physiological levels and remains resistant to non-specific inflammatory cell degradation.

The potential toxicity of the FAP probe was assessed by dosing rats with an intravenous (bolus) injection of 0.1 mg of the FAP3 probe for 7 days. There were no deaths, no clinical signs and no differences in body weights, body weight gains, food consumption, haematology, coagulation, clinical chemistry, organ weights and macroscopic and microscopic examinations compared to a control group of rats who had not received the FAP3 probe.

### Fluorescence Lifetime Imaging (FLIM) of NSCLC Tissue

Label free optical imaging modalities have the potential to characterise lung cancer using both optical endomicroscopy and fluorescence lifetime imaging (FLIM) systems. Here we used a clinically tractable fluorescence and FLIM imaging system, called KronoScan, compatible with bronchoscopy. To assess the ability of the system to detect both fluorescence and fluorescence intensity changes over time, varying concentrations of rhFAP were incubated with 5 μm FAP3 and imaged up to 40 minutes (data not shown). This demonstrated the ability of the FLIM system to detect changes in intensity and lifetime (within 5 minutes) with longer lifetime reflective of higher rhFAP concentrations.

Next, to assess if using FAP3 we could detect FAP specific cleavage (measured by a change in fluorescence intensity or lifetime) in biological samples we utilized ex vivo lung cancer specimens including two adenocarcinomas, two squamous cell carcinoma and one adenosquamous carcinoma. Representative images showing the intensity and FLIM measurements taken from the Fibre based FLIM imaging system are shown in FIG. 5A and the change in FLIM and intensity over time for this sample are shown in FIG. 5B further showing the increasing signals with time. Across all samples there was an increase in fluorescence intensity and a change to a longer FLIM signature following the addition of FAP3 over time, both of which were abrogated by VBP.

Summation of the data demonstrated significant FAP dependent increase in both intensity and longer lifetime across all samples (FIG. 5C). Assessing the data on a per cancer basis (FIG. 6) there are two interesting features to note—the FLIM signature of the intact probe becomes the dominant signature irrespective of the intrinsic autofluorescence FLIM and secondly, the rate of change varies amongst the different samples (CR68 and CR126 demonstrate maximum change within 10 minutes). This was most apparent for CR126 where the addition of the probe resulted in demonstration of immediate changes in FLIM ahead of the detectable fluorescence increase for the same sample (data not shown). Together, this demonstrates FAP3 can detect FAP specific activity in NSCLC using both changes in fluorescence intensity and lifetime and the higher rate of cleavage indicates presence of a higher concentration of FAP.

### CONCLUSIONS

We have developed an optical imaging probe capable of FAP imaging within physiological levels in NSCLC patient derived cancer associated fibroblasts and demonstrated changes in fluorescence intensity and lifetime in several patient samples. Furthermore, we have demonstrated specificity over PREP, a closely related endopeptidase to FAP, and MMPs and demonstrated stability in highly proteolytic conditions by testing against activated neutrophils. We have therefore developed a FAP specific optical imaging probe, which has potential applications in imaging of NSCLC as well as other FAP mediated inflammatory conditions.

### REFERENCES

[1] R. Liu, H. Li, L. Liu, J. Yu, and X. Ren, “Fibroblast activation protein,” Cancer Biol. Ther., vol. 13, no. 3, pp. 123-129, 2012.
[2] T. K. Kilvaer et al., “Tissue analyses reveal a potential immune-adjuvant function of FAP-1 positive fibroblasts in non-small cell lung cancer,” PLoS One, vol. 13, no. 2, pp. 1-17, 2018.
[3] P. S. Acharya, A. Zukas, V. Chandan, A.-L. A. Katzenstein, and E. Puré, “Fibroblast activation protein: a serine protease expressed at the remodeling interface in idiopathic pulmonary fibrosis.,” Hum. Pathol., vol. 37, no. 3, pp. 352-360, March 2006.
[4] A. J. Lay, H. E. Zhang, G. W. McCaughan, and M. D. Gorrell, “Fibroblast activation protein in liver fibrosis.,” Front. Biosci. (Landmark Ed., vol. 24, pp. 1-17, January 2019.
[5] S. Bauer et al., “Fibroblast activation protein is expressed by rheumatoid myofibroblast-like synoviocytes.,” Arthritis Res. Ther., vol. 8, no. 6, p. R171, 2006.
[6] J. Tillmanns et al., “Fibroblast activation protein alpha expression identifies activated fibroblasts after myocardial infarction.,” J. Mol. Cell. Cardiol., vol. 87, pp. 194-203, October 2015.
[7] J. D. Cheng, R. L. Dunbrack Jr., M. Valianou, A. Rogatko, R. K. Alpaugh, and L. M. Weiner, “Promotion of tumor growth by murine fibroblast activation protein, a serine protease, in an animal model,” Cancer Res., vol. 62, no. 16, pp. 4767-4772, 2002.
[8] J. A. Joyce and D. T. Fearon, “T cell exclusion, immune privilege, and the tumor microenvironment,” Science, vol. 348, no. 6230. pp. 74-80, 2015.
[9] H. Wang et al., “Downregulation of FAP suppresses cell proliferation and metastasis through PTEN/PI3K/AKT and Ras-ERK signaling in oral squamous cell carcinoma,” Cell Death Dis., vol. 5, no. 4, pp. 1-11, 2014.
[10] P. Mhawech-Fauceglia et al., “Stromal Expression of Fibroblast Activation Protein Alpha (FAP) Predicts Platinum Resistance and Shorter Recurrence in patients with Epithelial Ovarian Cancer,” Cancer Microenviron., vol. 8, no. 1, pp. 23-31, 2015.
[11] M. Shi et al., “Expression of fibroblast activation protein in human pancreatic adenocarcinoma and its clinicopathological significance,” World J. Gastroenterol., vol. 18, no. 8, pp. 840-846, 2012.
[12] T. K. Kilvaer et al., “Cancer Associated Fibroblasts in Stage I-IIIA NSCLC: Prognostic Impact and Their Correlations with Tumor Molecular Markers,” PLoS One, vol. 10, no. 8, August 2015.
[13]] E. Puré and R. Blomberg, “Pro-tumorigenic roles of fibroblast activation protein in cancer: back to the basics,” Oncogene, vol. 37, no. 32, pp. 4343-4357, 2018.
[14] Y. Liao, Y. Ni, R. He, W. Liu, and J. Du, “Clinical implications of fibroblast activation protein-alpha in non-small cell lung cancer after curative resection: a new predictor for prognosis,” J. Cancer Res. Clin. Oncol., vol. 139, no. 9, pp. 1523-1528, September 2013.
[15] E. J. Hamson, F. M. Keane, S. Tholen, O. Schilling, and M. D. Gorrell, “Understanding fibroblast activation protein (FAP): Substrates, activities, expression and targeting for cancer therapy,” PROTEOMICS Clin. Appl., vol. 8, no. 5-6, SI, pp. 454-463, June 2014.
[16] S. E. Poplawski et al., “Identification of selective and potent inhibitors of fibroblast activation protein and prolyl oligopeptidase,” J. Med. Chem., vol. 56, no. 9, pp. 3467-3477, 2013.
[17] A. Loktev et al., “A Tumor-Imaging Method Targeting Cancer-Associated Fibroblasts,” J. Nucl. Med., vol. 59, no. 9, pp. 1423-1429, September 2018.
[18] G. Larrinaga et al., “Increased prolyl endopeptidase activity in human neoplasia,” Regul. Pept., vol. 163, no. 1, pp. 102-106, 2010.
[19] V. J. Christiansen, K. W. Jackson, K. N. Lee, T. D. Downs, and P. A. McKee, “Targeting Inhibition of Fibroblast Activation Protein-α and Prolyl Oligopeptidase Activities on Cells Common to Metastatic Tumor Microenvironments,” Neoplasia, vol. 15, no. 4, pp. 348-358, 2013.
[20] J. Li et al., “An Activatable Near Infrared Fluorescent Probe for In Vivo Imaging of Fibroblast Activation Protein-alpha,” Bioconjug. Chem., vol. 23, no. 8, pp. 1704-1711, 2013.
[21] T. W. Bainbridge, D. R. Dunshee, N. M. Kljavin, N. J. Skelton, J. Sonoda, and J. A. Ernst, “Selective Homogeneous Assay for Circulating Endopeptidase Fibroblast Activation Protein (FAP),” Sci. Rep., vol. 7, no. 1, pp. 1-12, 2017.
[22] G. Pirovano, S. Roberts, S. Kossatz, and T. Reiner, “Optical imaging modalities: Principles and applications in preclinical research and clinical settings,” J. Nucl. Med., vol. 61, no. 10, pp. 1419-1427, 2020.
[23] C. Haslett, L. A. Guthrie, M. M. Kopaniak, R. B. Johnston, and P. M. Henson, “Modulation of multiple neutrophil functions by preparative methods or trace concentrations of bacterial lipopolysaccharide,” Am. J. Pathol., vol. 119, no. 1, pp. 101-110, 1985.
[24] J. Kargl et al., “Neutrophils dominate the immune cell composition in non-small cell lung cancer,” Nat. Commun., vol. 8, pp. 1-11, 2017.

1. A construct that is selectively cleaved by Fibroblast Activation Protein (FAP) comprising the peptide sequence:
A₋₂-A₋₁-A₀-A₊₁-A₊₂
wherein
A₋₂ is an D- or L-amino acid or derivative thereof;
A₋ is a D-amino acid, β-alanine ((β)Ala) or derivative thereof;
A₀ is proline (Pro) or derivate thereof;
A₊₁ is asparagine (Asn) or derivative thereof;
A₊₂ is a D- or L-amino acid or derivative thereof.

2. The construct according to claim 1, wherein
A₋₂ is selected from valine (Val), lysine (Lys) or derivative thereof;
A₋ is selected from D-alanine ((D)Ala), D-Serine ((D)Ser), D-threonine ((D)Thr), β-alanine ((β)Ala) or derivative thereof;
A₊₂ is selected from glutamine (Gln), glutamic acid (Glu), lysine (Lys) or derivative thereof.

3. The construct according to claim 1 comprising the peptide sequence:
A₋₃-A₋₂-A₋₁-A₀-A₊₁-A₊₂-A₊₃-A₊₄
wherein
A₋₂, A₋₁, A₀, A₊₁ and A₊₂ are as in claim 1;
A₋₃, A₊₃ and A₊₄ are a D- or L-amino acid or derivative thereof.

4. The construct according to claim 3, wherein
A₋₃ is selected from lysine (Lys), arginine (Arg), glutamine (Gln), asparagine (Asn) or derivative thereof;
A₊₃ is selected from glycine (Gly), cysteine (Cys) or derivative thereof;
A₊₄ is selected from lysine (Lys), arginine (Arg), glutamine (Gln), asparagine (Asn) or derivative thereof.

5. The construct according to claim 2, wherein the peptide sequence consists of Val-(D)Ser-Pro-Asn-Gln.

6. The construct according to claim 4, wherein the peptide sequence consists of Lys-Val-(D)Ser-Pro-Asn-Gln-Gly-Lys.

7. The construct according to claim 1, further comprising a solubilising moiety attached, bound or otherwise linked to one or more amino acids of the peptide sequence.

8. The construct according to claim 7, wherein the solubilising moiety is attached, bound or otherwise linked to N- or C-terminal amino acids.

9. The construct according to claim 7, wherein the amino acid attached, bound or otherwise linked to the solubilising moiety is in the D- or L-configuration.

10. The construct according to claim 7, wherein the solubilising moiety is a polyethylene glycol (PEG) derivative, optionally the solubilising moiety is PEG2.

11. (canceled)

12. The construct according to claim 6, wherein the construct comprises of the sequence Lys-Val-(D)Ser-Pro-Asn-Gln-Gly-[PEG2-(D)Lys]3-NH2.

13. The construct according to claim 1, wherein the construct further comprises at least one luminescent molecule, fluorescent molecule, quencher molecule, pH sensitive molecule, oxygen sensitive molecule or a combination thereof.

14. The construct according to claim 1, wherein the construct is a fluorescence resonance energy transfer (FRET) probe comprising a fluorescent moiety and a quencher moiety attached to the peptide sequence.

15. The construct according to claim 14, wherein the construct is a fluorescence energy transfer (FRET) probe comprising of a fluorophore moiety attached to A-n and a quencher moiety attached to A+n where n>2, or vice versa, optionally wherein the fluorophore moiety is attached to position A−3 and quencher moiety is attached to position A₊₄.

16. (canceled)

17. The construct according to claim 1 which is FAP3, as shown in FIG. 3a.

18. The construct according to claim 1, wherein the construct is resistant to cleavage or degradation at sites of inflammation.

19. The construct according to claim 18, wherein the construct is resistant to cleavage or degradation by enzymes and/or other inflammatory mediators produced by immune cells.

20. The construct according to claim 19, wherein the construct is resistant to cleavage or degradation by dipeptidyl peptidases and/or prolyl oligopeptidases, or by matrix remodelling enzymes.

21. (canceled)

22. A method of detecting a level of FAP in a sample from a body, the method comprises contacting a construct according to claim 1 with the body sample, ex vivo, in vivo, or in vitro and detecting any signal generated following cleavage of the construct by FAP.

23. The method according to claim 22, wherein the sample from the body comprises a cell containing sample obtained from a tumour or fibrotic tissue, or the sample from the body comprises bodily fluid, such as blood, serum, urine or plasma.

24. (canceled)


SEQUENCE LISTING (14 sequences)

SEQ ID NO: 1-2 - skipped (2 entries)

SEQ ID NO: 3 - protein, 5 aa, synthetic construct
  SITE 2: note = D-Serine
  VARIANT 1: note = Valine may be replaced by Lysine
  VARIANT 2: note = D-Serine may be replaced by D-Alanine, D-Threonine or beta-Alanine
  VARIANT 3: note = Proline may be replaced by D-Proline
  VARIANT 4: note = Asparagine may be replaced by D-Asparagine
  VARIANT 5: note = Glutamine may be replaced by Glutamic acid or Lysine
VSPNQ

SEQ ID NO: 4 - protein, 8 aa, synthetic construct
  SITE 3: note = D-Serine
  VARIANT 1: note = Lysine may be replaced by Arginine, Glutamine or Asparagine
  VARIANT 7: note = Glycine may be replaced by Cysteine
  VARIANT 8: note = Lysine may be replaced by Arginine, Glutamine or Asparagine
LVSPNQGL

SEQ ID NO: 5 - protein, 7 aa, synthetic construct
  SITE 1: note = Carboxyfluorescein-(Polyethylene glycol)2
  SITE 7: note = MethylRed
GPGPNQL

SEQ ID NO: 6 - protein, 7 aa, synthetic construct
  SITE 4: note = D-Proline
  SITE 5: note = D-Asparagine
  SITE 1: note = Carboxyfluorescein-(Polyethylene glycol)2
  SITE 7: note = MethylRed
GPGPNQL

SEQ ID NO: 7 - protein, 6 aa, synthetic construct
  SITE 6: note = D-Lysine
  SITE 1: note = MethylRed
  SITE 5: note = 5-Carboxyfluorescein
  SITE 6: note = (Polyethylene glycol)2
LAPSLL

SEQ ID NO: 8 - protein, 6 aa, synthetic construct
  SITE 2: note = D-Alanine
  SITE 6: note = D-Lysine
  SITE 1: note = MethylRed
  SITE 5: note = 5-Carboxyfluorescein
  SITE 6: note = (Polyethylene glycol)2
LAPSLL

SEQ ID NO: 9 - protein, 6 aa, synthetic construct
  SITE 2: note = D-Serine
  SITE 6: note = D-Lysine
  SITE 1: note = MethylRed
  SITE 5: note = 5-Carboxyfluorescein
  SITE 6: note = (Polyethylene glycol)2
LSPSLL

SEQ ID NO: 10 - protein, 6 aa, synthetic construct
  SITE 2: note = D-Threonine
  SITE 5: note = 5-Carboxyfluorescein
  SITE 1: note = MethylRed
  SITE 6: note = (Polyethylene glycol)2
  SITE 6: note = D-Lysine
LTPSLL

SEQ ID NO: 11 - protein, 6 aa, synthetic construct
  SITE 2: note = D-Tyrosine
  SITE 6: note = D-Lysine
  SITE 5: note = 5-Carboxyfluorescein
  SITE 1: note = MethylRed
  SITE 6: note = (Polyethylene glycol)2
LYPSLL

SEQ ID NO: 12 - protein, 6 aa, synthetic construct
  MOD_RES 2: note = beta-Alanine
  SITE 6: note = D-Lysine
  SITE 5: note = 5-Carboxyfluorescein
  SITE 1: note = MethylRed
  SITE 6: note = (Polyethylene glycol)2
LAPSLL

SEQ ID NO: 13 - protein, 9 aa, synthetic construct
  SITE 3: note = D-Serine
  SITE 9: note = D-Lysine
  SITE 8: note = 5-Carboxyfluorescein
  SITE 1: note = MethylRed
  SITE 9: note = (Polyethylene glycol)2
LVSPNQGLL

SEQ ID NO: 14 - protein, 9 aa, synthetic construct
  SITE 3: note = D-Serine
  SITE 9: note = D-Lysine
  SITE 8: note = 5-Carboxyfluorescein
  SITE 1: note = MethylRed
  SITE 9: note = (Polyethylene glycol)2
LVSPSQGLL